(19) United States
(12) Patent Application Publication
(10) Pub. No.: US 2008/0176264 A1
(43) Pub. Date: Jul. 24, 2008

(54) Osteoclast Culture Vessels and Methods of Use

(76) Inventor: Dale Greenwalt, Walkersville, MD (US)

Correspondence Address: BANNER & WITCOFF, LTD., 1100 13th STREET, N.W., SUITE 1200, WASHINGTON, DC, 20005-4051, US

(21) Appl. No.: 11/578,697

(22) PCT Filed: May 6, 2005
(86) PCT No.: PCT/US2005/016144

§ 371 Date: Oct. 22, 2007

Related U.S. Application Data
(60) Provisional application No. 60/568,274, filed on May 6, 2004.

(51) Int. Cl. C12Q 1/02; C12M 1/00

(52) U.S. Cl. 435/29; 435/289.1

(57) ABSTRACT

Osteoclast culture vessels comprising and adherent layer of bone particles for detecting osteoclast function.

This application claims the benefit of co-pending provisional application Ser. No. 60/568,274 filed May 6, 2004, which is incorporated herein by reference in its entirety.

Osteoporosis is a metabolic bone disease characterized by the deterioration of bone tissue and low bone mass density leading to bone fragility and risk of fracture. There is a continuing need in the art for rapid assays which can be used to identify drugs which reduce or prevent bone resorption.

### BRIEF DESCRIPTION OF THE FIGURES

FIG. 1. Graph showing time course of release of collagen peptides caused by differentiating primary human osteoclasts cultured in an OSTEOASSAY™ plate.

FIG. 2. Graphs comparing detection of collagen peptide release caused by primary human osteoclasts cultured either in an OSTEOASSAY™ plate or on a dentin disc. FIG. 2A, cells were cultured for 5 days and samples of supernatant were harvested after an additional 48 hours of culture and used in an EIA assay for the CTx telopeptide. FIG. 2B, cells were cultured for 5 days and samples of supernatant were harvested after an additional 24 hours of culture and used in an EIA assay for the helical peptide.

FIG. 3. Graph showing inhibition of bone resorption by calcitonin as assayed with an OSTEOASSAY™ plate.

FIG. 4. Graph showing inhibition of bone resorption by alendronate as assayed with an OSTEOASSAY™ plate.

### DETAILED DESCRIPTION OF THE INVENTION

The invention provides osteoclast culture vessels which comprise a thin adherent layer of bone particles for the culture of human or non-human osteoclasts or osteoclast precursors. Assays of osteoclast function can be performed easily by sampling the cell culture supernatant. Osteoclast culture vessels of the invention therefore provide a physiologically relevant and inexpensive substrate for conducting in vitro assays of osteoclast function, particularly bone resorption. Osteoclast culture vessels of the invention are well suited to high-throughput assays and are therefore useful for screening test compounds for an ability to affect osteoclast function, particularly to prevent or reduce bone resorption. Osteoclast culture vessels of the invention are compatible with commercial EIA kits and are superior to dentin discs in that results are obtained in days instead of after weeks of culture.

Vessel

Any vessel appropriate for culturing cells can be used as an osteoclast culture vessel of the invention, provided the culture surface of the vessel (the portion of the inner surface of the vessel onto which the cells are seeded) is a material to which bone particles can adhere. Appropriate vessels comprise at least one culture zone and include, for example, commercially available 4-, 6-, 8-, 12-, 24-, 96-, or 384-well plastic tissue culture plates or Petri dishes. Hydrophilic surfaces are particularly suitable. Useful materials include, but are not limited to, glass, polystyrene, polypropylene, polycarbonate, copolymers (e.g., ethylene vinylacetate copolymers), and the like.

Bone Particles

A culture zone of the osteoclast culture vessel comprises an adherent layer consisting essentially of bone particles. The layer preferably is substantially homogeneous, i.e., the depth of the layer is preferably no deeper than longest dimension of the largest bone particle in the layer. A perfectly uniform layer is not necessary, although a monolayer of bone particles is preferred.

Bone particles in the adherent layer can be any shape and need not be homogeneous in either shape or size within the adherent layer. Bone particles preferably are less than about ≦0.15 mm in their longest dimension (e.g., about 0.15, 0.13, 0.125, 0.12, 0.11, 0.1, 0.09, 0.08, 0.07, 0.06, 0.05, 0.04, 0.03, 0.02, 0.01 mm; or about 9, 8, 7, 6, 5, 4, 3, 2, or 1 μm). In some embodiments the size of the bone particles ranges from about 0.125 mm to about 1 μm.

“Bone” as used herein includes mammalian bone as well as dentin material obtained from horn, teeth, tusks, etc. Bone particles are preferably mammalian bone particles, especially bovine bone particles, and more preferably human bone particles. Non-human mammalian bone can be obtained from mammals such as cows, sheep, goats, horses, pigs, mice, rats, and guinea pigs. Bone can be ground to the desired particle size using well known methods, e.g., by grinding pieces of bone in a mill. Human bone typically is obtained from an organ procurement organization and can be ground to the desired particle size either by the organization or by the end-user. The Association of Organ Procurement Organizations maintains a list of organ procurement organizations which can provide suitable human bone fragments or particles.

OSTEOASSAY™ Plate

One embodiment of the invention is the 96-well OSTEOASSAY™ plate. The culture zones (wells) of the OSTEOASSAY™ plate contains an adherent layer comprising particles of native human bone which are ≦0.125 mm in their longest dimension. See Example 1.

Methods of Making Osteoclast Culture Vessels

To make osteoclast culture vessels of the invention, aliquots of an aqueous suspension of bone particles are dispensed into one or more culture zones of a vessel, preferably under sterile conditions. The aqueous suspension consists essentially of water and bone particles. It is helpful when making the aqueous suspension to stir it continuously to keep larger bone particles suspended. As those of skill in the art will appreciate, parameters such as the concentration of bone particles in the aqueous suspension and the amount of aqueous suspension dispensed in a culture zone depend on the size of the culture zone. Manipulation of these parameters is well within the skill of those in the art. After dispensing the aqueous suspension, the vessel can simply be air-dried, preferably under conditions which maintain sterility, e.g., in a laminar flow hood. No additional manipulation is required to make the bone particles adhere to the surface of a culture zone. At the end of the drying time, typically one to several days, the culture zone comprises an adherent layer of bone particles.

Methods of Assaying Osteoclast Function

Osteoclast culture vessels of the invention can be used to assay osteoclast function (e.g., osteoclast differentiation or bone resorption) using normal mammalian, preferably human, bone. Osteoclasts or osteoclast precursors are seeded on the adherent layer of bone particles and cultured in a suitable medium according to methods well known in the art. The culture medium can be assayed for the products of in vitro bone resorption and/or osteoclast differentiation. Assays can be carried out qualitatively or quantitatively and in either low- or high-throughput format.

Osteoclasts or Osteoclast Precursors

Assays of the invention can be used with any type of cell (either primary cells or established cell lines) which either causes bone resorption or which can differentiate into a cell which causes bone resorption. For example, cells such as osteoclasts or osteoclast precursors. “Osteoclasts” as used herein include differentiated osteoclasts as well as osteoclast-like cell lines. “Osteoclast precursors” as used herein include pre-osteoclasts, osteoclast progenitors, and osteoclast precursor cell lines. Purified cell populations (i.e., populations in which all or a majority of the cells are the desired type of cell) need not be used.

Osteoclasts or osteoclast precursors can be, for example, avian or mammalian. Avian osteoclasts or osteoclast precursors are disclosed, for example, in Collin-Osdoby et al., Methods Mol Med. 2003; 80:65-88; Collin-Osdoby et al., J Bone Miner Res. 2002 October; 17(10):1859-71. Suitable mammalian osteoclasts or osteoclast precursors include, but are not limited to, those of rodents, such as rat (Bushinsky, J Bone Miner Res. 1994 November; 9(11):1839-44) or mouse (Takahashi et al., Methods Mol Med. 2003; 80:129-44); rabbit (Coxon et al., Methods Mol Med. 2003; 80:89-99; Shimizu et al., Bone Miner. 1989 July; 6(3):261-75); non-human primates (Povolny & Lee, Exp Hematol. 1993 April; 21(4):532-7; Takahashi et al., J Bone Miner Res. 1987 August; 2(4):311-7); and humans (Sabokbar & Athanasou, Methods Mol Med. 2003; 80:101-11; Benito et al., Cytometry. 2002 Oct. 15; 50(5):261-6).

Methods of obtaining and culturing primary osteoclasts and primary osteoclast precursors are well known in the art. See, e.g., Chenu et al., Proc. Natl. Acad. Sci. USA 85, 5683-87, 1988; Hughes et al., J Clin Invest. 1989 June; 83(6): 1930-35; Roodman, Exp. Hematol. 27, 1229-41, 1999; Roux & Orcel, Arthritis Res. 2000; 2(6): 451-56; Toyosaki-Maeda et al., Arthritis Res. 2001; 3(5): 306-10.

A preferred source of human osteoclast precursors are those available from Cambrex Corporation (“POIETICS™ Osteoclast Precursors,” product no. 2T-110). Culture medium and additives for culturing osteoclast precursor cells can also be obtained from Cambrex Corporation (e.g, PT-8001; PT-8201; PT-9501).

Other suitable cells include, but are not limited to, MOCP-5 osteoclast precursors (Chen & Li, J Bone Miner Res. 1998 July; 13(7):1112-23); osteoclast-inductive and osteoclastogenic cell lines from the H-2K^{b}tsA58 transgenic mouse (Chambers et al., Proc. Natl. Acad. Sci. USA 90, 5578-82, 1993); and the immortalized osteoclast (OCL) precursor cell line derived from mice doubly transgenic for bcl-XL and large T antigen (Hentunen et al., Endoctinology 140, 2954-61, 1999). Pre-osteoclast and osteoclast-like cell lines also can be used. See, for example, Fiorelli et al., Proc. Natl. Acad. Sci. USA 92, 2672-76, 1995; Miyamoto & Suda, Keio J. Med. 52, 1-7, 2003; Arai et al., J. Exp. Med. 190, 1741-54, 1999; Espinosa et al., J. Cell Sci. 115, 3837-48, 2002; Mbalaviele et al., J. Cell Biol. 141, 1467-76, 1998; Thomas et al., Endocrinol. 140, 4451-58, 1999; Quinn et al., Endocrinol. 139, 4424-27, 1998; Itoh et al., Endocrinol. 142, 3656-62, 2001; and Ragab et al., Am. J. Physiol. Cell Physiol. 283, C679-C687, 2002. See also MacDonald et al., J Bone Miner Res. 1986 April; 1(2):227-33.

The selection of an appropriate culture medium for a given cell type, as well as other culture conditions such as temperature and percent CO₂, is well within the skill of those in the art (see, for example, ANIMAL CELL CULTURE, R. I. Freshney, ed., 1986).

Culture times can be varied according to the type of cells cultured. For example, if osteoclast precursors are used, they can be cultured for 4-5 days before an assay is carried out so that the cells can differentiate into functional osteoclasts. If osteoclasts or osteoclast-like cell lines are used, assays can be carried out after seeded cells become adherent, preferably 24 hours after seeding.

Testing for Osteoclast Function

Osteoclast function can be assessed either by detecting bone resorption or osteoclast differentiation. Bone resorption can be detected in a variety of ways, including detecting calcium or collagen (or collagen fragments) released into the culture medium.

Calcium can be detected using any method known in the art. Colorimetric assays for calcium are disclosed, for example, in Alatas et al., Clin Biochem. 2002 June; 35(4):293-6; Lin et al., J Pharm Biomed Anal. 1999 December; 21(5):931-43; Mann & Green, Ann Clin Biochem. 1988 July; 25 (Pt 4):444; and Corns, Ann Clin Biochem. 1987 November; 24 (Pt 6):591-7. Fluorescence-based calcium assays are disclosed, for example, in Witte et al., J Biomol Screen. 2002 October; 7(5):466-75; Zhang et al., J Biomol Screen. 2003 October; 8(5):571-7; Chambers et al., Comb Chem High Throughput Screen. 2003 June; 6(4):355-62; Princen et al., Cytometry. 2003 January; 51A(1):35-45; and Sullivan et al., Methods Mol Biol. 1999; 114:125-33. Commercially available kits can be used, such as the QUANTICHROM™ (BioCan) kit.

Collagen or collagen fragments are conveniently detected using an antibody which specifically binds to a collagen epitope. As is well known in the art, either the collagen antibody or a secondary antibody which binds to the collagen antibody can be labeled with a detectable label, such as a fluorescent, luminescent, colorimetric, or radioactive label. A variety of commercially available EIA assays and kits can be used to detect collagen, such as the METRA™ Helical Peptide EIA kit (Quidel), which detects type I collagen; the Serum CROSSLAPS® ELISA (Nordic Bioscience), which detects an epitope localized in the C-terminal telopeptide of the collagen type I; and OSTEOMARK® NTx (Ostex), which detects cross-linked N-telopeptides of bone type I collagen.

Osteoclast differentiation can be assessed by detecting products of bone resorption, as described above, or by detecting biochemical markers of osteoclast differentiation, such as tartrate-resistant acid phosphatase type 5b (“TRAP” or “TRAP 5b”) (Minkin, Calcif Tissue Int 1982; 34:285-90), which is secreted into the culture medium. TRAP assays are well known and include cytochemical stains (calorimetric), enzyme assays (e.g., fluorescent, luminescent or colorimetric), and immunoassays (e.g., calorimetric or fluorescent). Other markers, such as the vitronectin receptor (integrin α_{V}β₃), can be detected by immunochemical methods as is known in the art.

Test Compounds

Test compounds can be added at any point during the culture of osteoclasts or osteoclast precursors to screen the test compounds for the ability to affect osteoclast function. Test compounds can be any pharmacologic agents already known in the art or can be compounds previously unknown to have any pharmacological activity. Test substances can be naturally occurring or synthesized in the laboratory. They can be isolated from microorganisms, animals, or plants, or can be produced recombinantly or synthesized by chemical methods known in the art.

Test compounds can be obtained from compound libraries. Methods of generating combinatorial libraries of test compounds are known in the art and include, but are not limited to, formation of “biological libraries,” spatially addressable parallel solid phase or solution phase libraries, synthetic library methods requiring deconvolution, the “one-bead one-compound” library method, and synthetic library methods using affinity chromatography selection. See, e.g., DeWitt et al., Proc. Natl. Acad. Sci. U.S.A. 90, 6909, 1993; Erb et al., Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061, 1994; Gallop et al., J. Med. Chem. 37, 1233, 1994; and Lam, Anticancer Drug Des. 12, 145, 1997.

Test compounds can be presented to cells, for example, in solution (Houghten, Biotechniques 13, 412-21, 1992), on beads (Lam, Nature 354, 82-84, 1991), in plasmids (Cull et al., Proc. Natl. Acad. Sci. U.S.A. 89, 1865-69, 1992), or in phage (Scott & Smith, Science 249, 386-90, 1990; Devlin, Science 249, 404-06, 1990; Cwirla et al., Proc. Natl. Acad. Sci. 97, 6378-82, 1990; Felici, J. Mol. Biol. 222, 301-10, 1991; and U.S. Pat. No. 5,223,409). For assays of the invention, test compounds are most conveniently added to the culture medium. This can be done either after the cells are placed in the osteoclast culture vessels or before.

Kits

The invention also provides kits which comprise one or more osteoclast culture vessels of the invention and instructions for carrying out one or more embodiments of the assays disclosed herein. The kits can contain other components, such as reagents for detecting bone resorption, buffers, osteoclasts or osteoclast precursors, culture medium, growth factors, etc.

All patents, patent applications, and references cited in this disclosure are expressly incorporated herein by reference in their entireties. The above disclosure generally describes the present invention. A more complete understanding can be obtained by reference to the following specific examples, which are provided for purposes of illustration only and are not intended to limit the scope of the invention.

### EXAMPLE 1

This example describes the evaluation of an OSTEOASSAY™ bone plate as an alternative to existing commercial substrates for use in assays of in vitro bone resorption.

OSTEOASSAY™ plates, which contain adherent particles (≦0.125 mm dimension) of native human bone, were seeded with 10,000 primary human osteoclast precursors (“Poietics™ Osteoclast Precursors,” product no. 2T-110, Cambrex Corporation) per well in medium containing M-CSF+/−soluble RANK ligand and cultured at 37° C. for 5-7 days. Culture media were changed on day 5, 6 or 7 and the cultures were continued for another 1 to 3 days.

The release into the culture supernatants of collagen type I peptides from the bone particles was measured quantitatively by enzyme immunoassay (EIA). Three different commercially available assay kits for the measurement of resorption-specific collagen resorption fragments were evaluated: the METRA™ Helical Peptide EIA kit (Quidel), the Serum CROSSLAPS® ELISA (Nordic Bioscience) kit, and the OSTEOMARK® NTx (Ostex) kit. In some experiments, the OSTEOASSAY™ plate was compared to dentin discs (ALPCO Diagnostics). In other assays, primary human osteoclast precursors were exposed to inhibitors of osteoclast function (alendronate or calcitonin) for 48-72 hours and then assayed for bone resorptive activity.

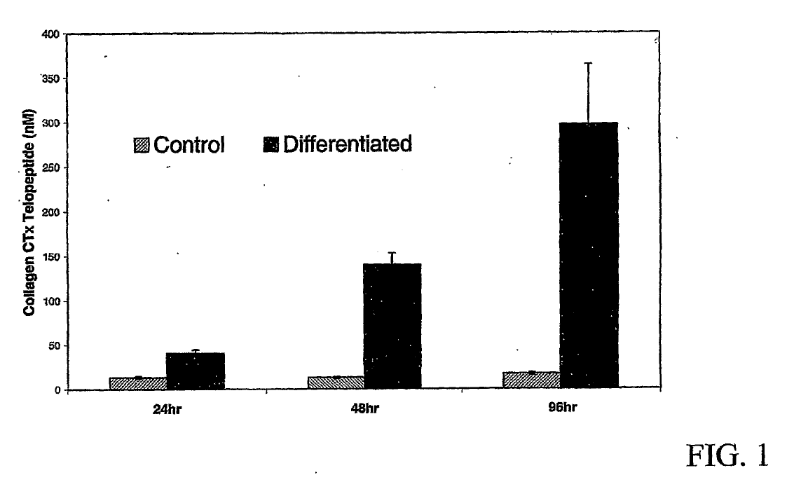

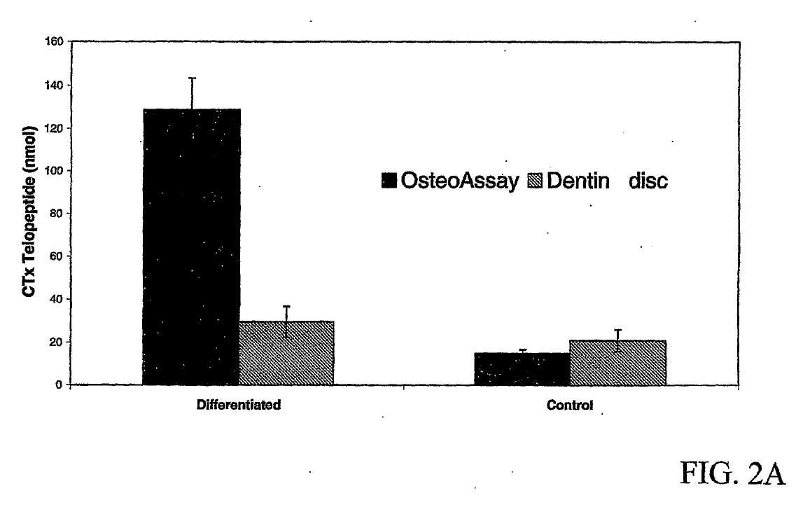

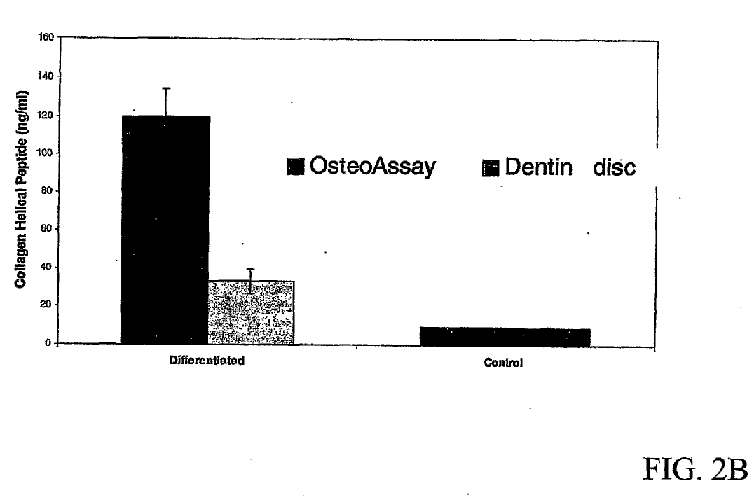

The release of collagen resorption products in supernatants of osteoclast precursors differentiated in OSTEOASSAY™ plates was substantially linear with time (FIG. 1). The signal-to-background (S:N) ratios (differentiated cells relative to undifferentiated precursors) also increase with time to >10 after a 48 hour assay period. Significant differences in S:N values and background levels were observed when comparing three commercially available EIA kits for the measurement of collagen resorption peptides. The S:N ratio of the Quidel EIA kit>the Nordic Bioscience EIA kit>the Ostex EIA kit (FIGS. 2A, 2B).

The ability of primary human osteoclasts to resorb bone was measured with both the OSTEOASSAY™ plate and with dentin discs (FIGS. 2A, 2B). In a 24 hour period (day 5 to day 6), the amount of collagen peptide released from the dentin discs was nearly 4-fold less than that released from the OSTEOASSAY™ plate as assayed with the Quidel EIA kit. Similarly, when the comparison was done over a 2-day period with the Nordic Bioscience EIA kit, the dentin disc released only 23% of the amount of collagen peptide released from the human bone chips in the OSTEOASSAY™ plate.

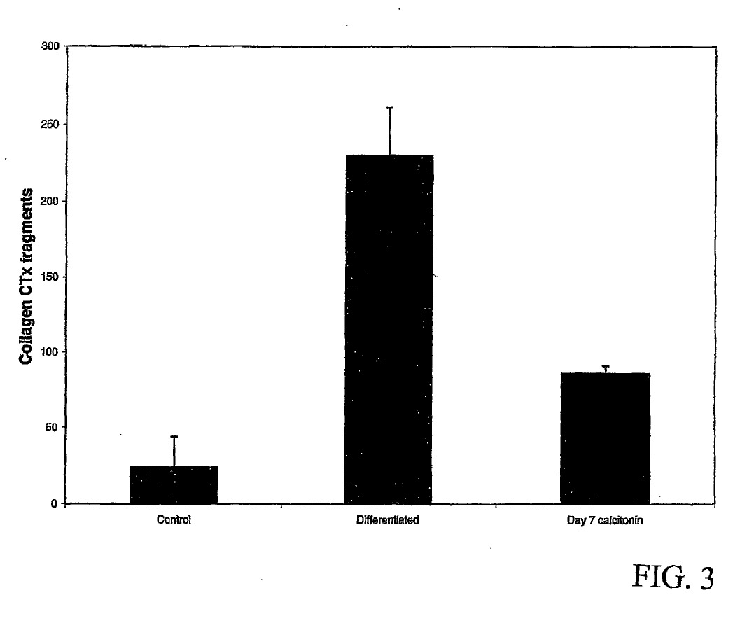

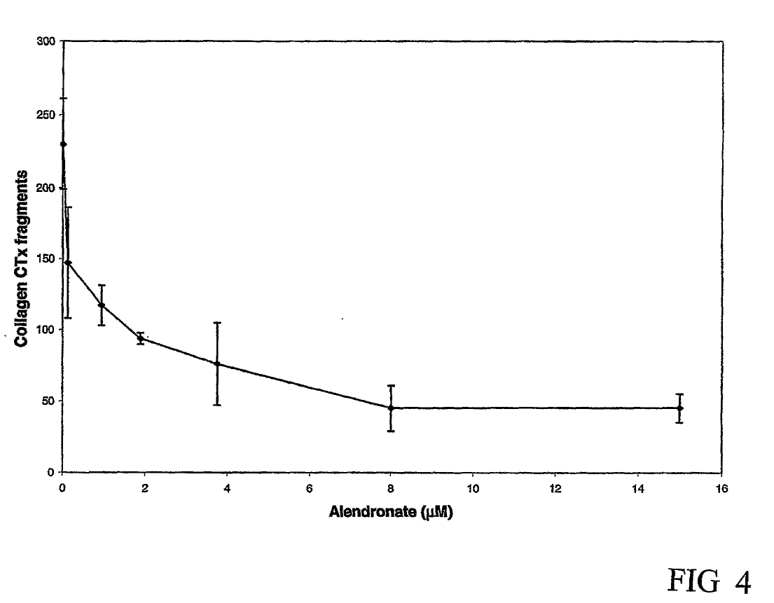

Primary human osteoclast precursors were seeded at 10,000 cells/well and cultured in medium containing M-CSF and soluble RANK ligand. After 5 days of culture, the medium was renewed +/−1 nM calcitonin or +/−various concentrations of alendronate. After an additional 2 days of culture, supernatants from each well were assayed for the presence of CTx collagen resorption product. FIG. 3 shows that calcitonin effectively inhibits in vitro bone resorption in an OSTEOASSAY™ plate. FIG. 4 shows that the IC₅₀ value for alendronate was about 1 μM. The coefficient of variation of assays done with the OSTEOASSAY™ plate averaged 12% with a Z′ value >0.5.

1. An osteoclast culture vessel comprising at least one culture zone which comprises an adherent layer consisting essentially of bone particles which are less than about 0.15 mm in longest dimension.

2. The osteoclast culture vessel of claim 1 wherein the bone particles are less than about 0.125 mm in longest dimension.

3. The osteoclast culture vessel of claim 1 wherein the bone particles are between about 1 μm and about 0.125 mm in longest dimension.

4. The osteoclast culture vessel of claim 1 which comprises a plurality of culture zones.

5. The osteoclast culture vessel of claim 1 wherein the bone particles comprise mammalian bone particles.

6. The osteoclast culture vessel of claim 1 wherein the bone particles comprise human bone particles.

7. The osteoclast culture vessel of claim 1 wherein the bone particles comprise bovine bone particles.

8. The osteoclast culture vessel of claim 1 wherein the adherent layer is substantially homogeneous.

9. The osteoclast culture vessel of claim 1 wherein the adherent layer is a monolayer of bone particles.

10. The osteoclast culture vessel of claim 1 wherein the at least one culture zone further comprises culture medium and a population of osteoclasts or osteoclast precursors which overlie the adherent layer.

11. The osteoclast culture vessel of claim 10 wherein the population of osteoclasts or osteoclast precursors comprises human osteoclasts or human osteoclast, precursors.

12. The osteoclast culture vessel of claim 10 wherein the culture medium comprises a test compound.

13. A method of detecting osteoclast function comprising:
examining culture medium of an osteoclast culture vessel to detect:
(a) a moiety which reflects resorption of the bone particles; or
(b) a marker of osteoclast differentiation,
wherein the osteoclast culture vessel comprises at least one culture zone which comprises an adherent layer consisting essentially of bone particles which are less than about 0.15 mm in longest dimension and wherein the at least one culture zone further comprises culture medium and a population of osteoclasts or osteoclast precursors which overlie the adherent layer.

14. The method of claim 13 further comprising culturing osteoclast or osteoclast precursors on the adherent layer of bone particles of the osteoclast culture vessel in the presence of the culture medium.

15. The method of claim 13 wherein the culture medium is examined to detect a marker of osteoclast differentiation and the marker is tartrate-resistant acid phosphatase type 5b (TRAP) secretion or TRAP activity.

16. The method of claim 13 wherein the culture medium is examined to detect a moiety which reflects resorption of the adherent bone particles and the moiety is calcium.

17. The method of claim 13 wherein the culture medium is examined to detect a moiety which reflects resorption of the adherent bone particles and the moiety is collagen or a collagen fragment.

18. The method of claim 13 further comprising contacting the osteoclast or osteoclast precursors with a test compound before the step of examining.

19. The method of claim 13 wherein the step of examining is carried out using an immunoassay.

20. The method of claim 13 wherein the step of examining is carried out using a colorimetric assay.

21. The method of claim 13 wherein the step of examining is carried out using an enzymatic assay.

22. The method of claim 13 wherein the population of osteoclast or osteoclast precursors comprises human osteoclast or human osteoclast precursors.

23. A method of making an osteoclast culture vessel, comprising:
dispensing into a culture zone of a vessel a composition consisting essentially of water and bone particles which are less than about 0.15 mm in longest dimension, whereby upon evaporation of the water the culture zone comprises an adherent layer of the bone particles.

24. The method of claim 23 wherein the bone particles are less than about 0.125 mm in longest dimension.

25. The method claim 23 wherein the bone particles are between about 1 μm and about 0.125 mm in longest dimension.

26. The method of claim 23 wherein the vessel comprises a plurality of culture zones.

27. The method of claim 23 wherein the bone particles comprise mammalian bone particles.

28. The method of claim 23 wherein the bone particles comprise human bone particles.

29. The method of claim 23 wherein the bone particles comprise bovine bone particles.

30. An osteoclast culture vessel made by the method of claim 23.
